(19) United States
(12) Patent Application Publication
(10) Pub. No.: US 2018/0312873 A1
(43) Pub. Date: Nov. 1, 2018

(54) METHOD AND SYSTEMS FOR HIGH THROUGHPUT SINGLE CELL GENETIC MANIPULATION

(71) Applicant: 10X Genomics, Inc., Pleasanton, CA (US)

(72) Inventor: Xinying ZHENG, Mountain View, CA (US)

(73) Assignee: 10X Genomics, Inc., Pleasanton, CA (US)

(21) Appl. No.: 15/769,609

(22) PCT Filed: Oct. 17, 2016
(86) PCT No.: PCT/US16/57395

§ 371 Date: Apr. 19, 2018

Related U.S. Application Data
(60) Provisional application No. 62/243,917, filed on Oct. 20, 2015.

(51) Int. Cl. C12N 15/88

(52) U.S. Cl. CPC C12N 15/88

(57) ABSTRACT

Provided herein are methods and systems for introducing nucleic acid manipulation agents into single cells. Such high throughput delivery of nucleic acid manipulation reagents into single cells and subsequent genetic manipulation of such cells allow for large scale genetic analysis that can be useful, for example, for the study of biological pathways and drug target discovery.

### CROSS-REFERENCE TO RELATED APPLICATIONS

This application claims the benefit of U.S. Provisional Application No. 62/243,917, filed Oct. 20, 2015, which is hereby incorporated by reference in its entirety for all purposes.

### BACKGROUND OF THE INVENTION

Advances in the development of nucleic acid manipulation reagents have allowed for simple and efficient manipulation of nucleic acids (e.g., DNA and RNA) in target cells. RNA interference (RNAi) reagents such as single interference RNAs (siRNAs) and short hairpin RNAs (shRNAs) allow for the cleavage, degradation and/or translation repression of target RNAs with adequate complementary sequence. The development of CRISPR reagents have provide DNA-encoded, RNA mediated, DNA- or RNA-targeting sequence specific targeting. CRISPR systems can be used to generate small insertions or deletions that cause impactful and inactivating mutations in target nucleic acids. In addition, CRISPR reagents have also been used for the precise insertion of donor DNA into a target cell genome. Such nucleic acid manipulation reagents have enabled researchers to precisely manipulate specific genomic elements and facilitate the function elucidation of target nucleic acids in biology and diseases.

Nucleic acid manipulation reagents have great potential for use in high throughput applications such as genome-wide mutation screens, drug target discovery and the large scale production of transgenic cells and organisms for research and commercial purposes. As new nucleic acid manipulation reagents for high throughput purposes are developed, however, there also is a need for the development of systems and methods for the high throughput introduction of these reagents into cells. Standard array screening methods require arranging cells and nucleic acid manipulation reagents into multiwell plates with a single reagent per well. Such screens oftentimes require special facilities that use automation for the handling of many plates. As such, large scale applications of these nucleic acid manipulation reagents can be expensive and time consuming processes. Accordingly, there is a need for new systems and methods for the high throughput delivery of nucleic acid manipulation reagents.

### SUMMARY OF THE INVENTION

Provided herein are compositions, systems and methods for the delivery of reagents into individual cells.

In a first aspect, provided herein is a method of delivering reagents into individual cells. Such a method includes without limitation the steps of: (a) providing a plurality of capsules, wherein the capsules comprise reagents for altering expression of at least one gene product in a cell; (b) delivering the capsules into discrete partitions, wherein the discrete partitions further comprise individual cells; and (c) causing the capsules to release their contents into the discrete partitions under conditions enabling uptake of the reagents by the individual cells, thereby delivering the reagents into the individual cells.

In some embodiments and in accordance with the above, the reagents for altering expression of at least one gene product includes (i) a first regulatory element operably linked to at least one nucleotide sequence encoding a CRISPR system guide RNA that hybridizes with a target sequence in a DNA molecule within the individual cells, and (ii) a second regulatory element operably linked to a nucleotide sequence encoding a RNA-guided nuclease or an RNA-guided nuclease fusion protein. In some embodiments, the components (i) and (ii) are located on same or different vectors, and the RNA-guided nuclease and the guide RNA do not naturally occur together.

In an exemplary embodiment, the second regulatory element is operably linked to a nucleotide sequence encoding a RNA-guided nuclease. In such an embodiment, the guide RNA targets the target sequence and the RNA-guided nuclease cleaves the DNA molecule, whereby expression of the at least one gene product is altered. In some embodiments, the reagents for altering gene expression further includes a donor nucleic acid that is inserted into the DNA molecule following cleavage of the DNA molecule by the RNA-guided nuclease.

In another exemplary embodiment, the second regulatory element is operably linked to a nucleotide sequence encoding a deactivated RNA-guided nuclease, whereby the guide RNA targets the target sequence and the deactivated RNA-guided nuclease interferes with the transcription of a nucleic acid encoding the at least one gene product, whereby expression of the at least one gene product is altered.

In yet another exemplary embodiment, the second regulatory element is operably linked to a nucleotide sequence encoding an RNA-guided nuclease fusion protein, whereby the guide RNA targets the target sequence and the RNA-guided nuclease fusion protein interferes with the expression of the at least one gene product, whereby expression of the at least one gene product is altered. In some instances, the RNA-guided nuclease fusion protein includes a deactivated RNA-guided nuclease and a transcription activator or a transcription repressor. In some instances, the nuclease fusion protein includes a deactivated RNA-guided nuclease and an epigenetic modifier.

In certain embodiments and in accordance with the above, the RNA-guided nuclease is a Cas9 protein or a Cpf1 protein.

In certain embodiments and in accordance with the above, the capsules are configured to release their contents upon the application of a stimulus. In some embodiments, the stimulus is selected from a chemical stimulus, an electrical stimulus, a thermal stimulus, a magnetic stimulus, a change in pH, a change in ion concentration, reduction of disulfide bonds, a photostimulus, and combinations thereof. In some embodiments, the stimulus is a thermal stimulus.

In further embodiments and in accordance with any of the above, the plurality of capsules includes about 100-100,000 different reagents for altering expression of at least one gene product, such that different individual cells receive different reagents.

In further embodiments and in accordance with any of the above, the capsules further include one or more additives for compatibility of the capsules or their contents with the individual cells. In some embodiments, the one or more additives include a transfection agent.

In certain embodiments and in accordance with any of the above, the reagents for altering expression of at least one gene product further include oligonucleotides that include a nucleic acid barcode sequence. In some of these embodiments, different individual cells receive different nucleic acid barcode sequences.

In some embodiments, the reagents for altering expression of at least one gene product described above further comprise a pair of Cas9 nickases or Cas9 fusion proteins that improve specificity of the CRISPR system as compared to when RNA-guided nucleases are used.

In certain embodiments and in accordance with any of the above, the target sequence has few or no close relatives within the cellular genome.

In further embodiments and in accordance with any of the above, the reagents for altering expression of at least one gene product further include agents that increase the frequency of homologous recombination in the cell by repressing genes involved in non-homologous end-joining (NHEJ) pathway. In some embodiments, these agents comprise a Cas9 nuclease or a nuclease-null Cas9 protein encoded with the at least one nucleotide sequence encoding a CRISPR system guide RNA.

In yet further embodiments and in accordance with any of the above, the guide RNA further comprises a spacer that is identical to a targeted protospacer sequence within the cell's genome.

In another exemplary embodiment and in accordance with any of the above, one or both of the first and second regulatory elements is an inducible promoter. In some embodiments, the inducible promoter is selected from the group consisting of a light-inducible, a heat-inducible and a chemical inducible promoter.

In a second aspect, provided herein is a method for delivering a reagent to a cell. This method includes without limitation the steps of (a) providing the reagent releasably coupled to a microcapsule; (b) separating the microcapsule into a discrete partition, wherein the discrete partition further comprises an individual cell; and (c) releasing the reagent under conditions that enable uptake of the reagent into the cell.

In some embodiments of this second aspect, the reagent includes a vector encoding at least one of a RNA-guided nuclease, Clustered Regularly Interspaced Short Palindromic Repeats (CRISPRs), or CRISPR guide RNA capable of hybridizing with one or more target sequences in a DNA molecule of the cell, and one or more condition-inducible promoters. In certain embodiments, the reagent includes (i) a first regulatory element operable in a eukaryotic cell operably linked to at least one nucleotide sequence encoding a CRISPR system guide RNA that hybridizes with a target sequence within the cell, and (ii) a second regulatory element operable in a eukaryotic cell operably linked to a nucleotide sequence encoding a RNA-guided nuclease. In some embodiments, components (i) and (ii) are located on same or different vectors, and, the RNA-guided nuclease and the guide RNA do not naturally occur together.

In an exemplary embodiment of this second aspect, the second regulatory element is operably linked to a nucleotide sequence encoding a RNA-guided nuclease, whereby the guide RNA targets the target sequence and the RNA-guided nuclease cleaves the DNA molecule, whereby expression of the at least one gene product is altered. In some embodiments, the reagents for altering gene expression further include a donor nucleic acid that is inserted into the DNA molecule following cleavage of the DNA molecule by the RNA-guided nuclease.

In another exemplary embodiment of this second aspect, the second regulatory element is operably linked to a nucleotide sequence encoding a deactivated RNA-guided nuclease, whereby the guide RNA targets the target sequence and the deactivated RNA-guided nuclease interferes with the transcription of a nucleic acid encoding the at least one gene product, whereby expression of the at least one gene product is altered.

In yet another exemplary embodiment of this second aspect, the second regulatory element is operably linked to a nucleotide sequence encoding a RNA-guided nuclease fusion protein, whereby the guide RNA targets the target sequence and the RNA-guided nuclease fusion protein interferes with the expression of the least one gene product, whereby expression of the at least one gene product is altered. In some embodiments, the RNA-guided nuclease fusion protein includes a deactivated RNA-guided nuclease and a transcription activator or a transcription repressor. In certain embodiments, the nuclease fusion protein comprises a deactivated RNA-guided nuclease and an epigenetic modifier.

In some embodiments of this second aspect, the RNA-guided nuclease is a Cas9 protein. In other embodiments, the RNA-guided nuclease is a Cpf1 protein. In some embodiments, the vector or vectors are capable of stable integration into the cell's genome.

In further embodiments, the releasing step includes applying a stimulus to the microcapsule to release the reagent. In some embodiments, the stimulus is selected from a chemical stimulus, an electrical stimulus, a thermal stimulus, a magnetic stimulus, a change in pH, a change in ion concentration, reduction of disulfide bonds, a photostimulus, and combinations thereof.

In some embodiments, the uptake of the reagent into the cell is facilitated by electroporation.

In further embodiments, the microcapsule further comprises one or more additives to improve compatibility of the reagent for uptake into the cell. In some embodiments, the one or more additives includes a transfection agent.

In some embodiments, the microcapsule includes a member selected from a droplet in an emulsion and a crosslinked polymer.

In further embodiments, the microcapsule includes a bead. In certain embodiments, the bead is a gel bead.

In yet further embodiments, the microcapsule further includes a population of nucleic acid barcode sequences releasably coupled thereto, where the barcode sequences substantially all include the same barcode sequence. In some embodiments, the barcode sequences further include a hairpin sequence.

In still further embodiments, the reagent further includes a pair of Cas9 nickases or Cas9 fusion proteins that improve specificity of the CRISPR system as compared to when RNA-guided nucleases are used.

In a third aspect, provided herein is a method for altering gene expression in a plurality of cells. The method includes without limitation the steps of (a) providing a plurality of capsules, where capsules include reagents for altering expression of at least one gene product, the reagents include an engineered, non-naturally occurring Clustered Regularly Interspaced Short Palindromic Repeats (CRISPR) system; (b) delivering the capsules into discrete partitions containing individual cells; (c) providing a stimulus to cause the capsules to release their contents under conditions such that reagents are delivered into the individual cells. In some embodiments, the CRISPR system includes one or more vectors that include (i) a first regulatory element operably linked to at least one nucleotide sequence encoding a CRISPR-Cas system guide RNA capable of hybridizing with a target sequence in a DNA molecule of the cell, and (ii) a second regulatory element operably linked to a nucleotide sequence encoding a RNA-guided nuclease, wherein components (i) and (ii) are located on same or different vectors of the system. In this aspect, subsequent to application of the stimulus, the guide RNA hybridizes to the target sequence and the RNA-guided nuclease cleaves the DNA molecule containing the target sequence, whereby expression of the at least one gene product is altered. In some embodiments of this aspect, the reagents for altering gene expression further include a donor nucleic acid that is inserted into the DNA molecule following cleavage of the DNA molecule by the RNA-guided nuclease.

In a fourth aspect, provided herein is a method for altering gene expression in a plurality of cells. The method includes without limitation the steps of (a) providing a plurality of capsules, where capsules include reagents for altering expression of at least one gene product, the reagents include an engineered, non-naturally occurring Clustered Regularly Interspaced Short Palindromic Repeats (CRISPR) system; (b) delivering the capsules into discrete partitions containing individual cells; (c) providing a stimulus to cause the capsules to release their contents under conditions such that reagents are delivered into the individual cells. In some embodiments, the CRISPR system includes one or more vectors that include (i) a first regulatory element operably linked to at least one nucleotide sequence encoding a CRISPR-Cas system guide RNA capable of hybridizing with a target sequence in a DNA molecule of the cell, and (ii) a second regulatory element operably linked to a nucleotide sequence encoding a deactivated RNA-guided nuclease, wherein components (i) and (ii) are located on same or different vectors of the system. In this aspect, subsequent to application of the stimulus, the guide RNA hybridizes to the target sequence and the deactivated RNA-guided nuclease interferes with the transcription of a nucleic acid encoding the at least one gene product, whereby expression of the at least one gene product is altered.

In a fifth aspect, provided herein is a method for altering gene expression in a plurality of cells. The method includes without limitation the steps of (a) providing a plurality of capsules, where capsules include reagents for altering expression of at least one gene product, the reagents include an engineered, non-naturally occurring Clustered Regularly Interspaced Short Palindromic Repeats (CRISPR) system; (b) delivering the capsules into discrete partitions containing individual cells; (c) providing a stimulus to cause the capsules to release their contents under conditions such that reagents are delivered into the individual cells. In some embodiments, the CRISPR system includes one or more vectors that include (i) a first regulatory element operably linked to at least one nucleotide sequence encoding a CRISPR-Cas system guide RNA capable of hybridizing with a target sequence in a DNA molecule of the cell, and (ii) a second regulatory element operably linked to a nucleotide sequence encoding a a RNA-guided nuclease fusion protein, wherein components (i) and (ii) are located on same or different vectors of the system. In this aspect, subsequent to application of the stimulus, the guide RNA hybridizes to the target sequence and the RNA-guided nuclease fusion protein interferes with the expression of the at least one gene product, whereby expression of the at least one gene product is altered. In some embodiments, the RNA-guided nuclease fusion protein includes a deactivated RNA-guided nuclease and a transcription activator or a transcription repressor. In certain embodiments, the nuclease fusion protein includes a deactivated RNA-guided nuclease and an epigenetic modifier.

In some embodiments of the third, fourth and fifth aspects, the RNA-guided nuclease is a Cas9 protein or a Cpf1 protein.

In some embodiments of the third, fourth and fifth aspects, the different capsules include guide RNAs that are capable of hybridizing to different target sequences within the individual cells, such that expression of different gene products is altered in different cells.

In further embodiments, the plurality of capsules includes about 500 to about 100,000 capsules. In some embodiments, the plurality of capsules includes about 10,000 to about 50,000 capsules. In other embodiments, the plurality of capsules comprises about 15,000 to about 30,000 capsules, where only a single capsule is delivered into each discrete partition.

In yet further embodiments, the capsules include a droplet in an emulsion. In other embodiments, the capsules include a polymer gel. In some embodiments, the polymer gel is a polyacrylamide. In further embodiments, the capsules include a gel bead.

### BRIEF DESCRIPTION OF THE DRAWINGS

FIG. 1 provides a schematic illustration of a microfluidic device for delivery of nucleic acid manipulation reagents into partitions that include single cells, as described herein.

### I. OVERVIEW

This disclosure provides methods, compositions, and systems useful for reagent delivery into single cells. In particular, the methods, compositions, and systems provided herein allow for the high throughput delivery of reagents for the manipulation of one or more target nucleic acids in individual cells. In some instances, such nucleic acid manipulation reagents alter the expression of a gene product encoded by the target nucleic acid. Such high throughput delivery of nucleic acid manipulation reagents into single cells and subsequent genetic manipulation of such cells allow for large scale genetic analysis that can be useful, for example, for the study of biological pathways and drug target discovery. Moreover, such high throughput gene editing can facilitate the production of genetic plants and animals and the development of cell-based therapeutics.

In general, provided herein is a method for delivery of nucleic acid manipulation reagents into individual cells. The method includes the step of providing a plurality of capsules, each carrying reagents for nucleic acid manipulation in individual cells. The provided capsules are delivered into discrete partitions that include one or more cells. After the delivery of the capsules into the partitions that include the cells, the capsules are caused to release their contents into the discrete partitions, generally through the use of a stimulus. In the presence of uptake reagents (e.g., transfection reagents or electroporation buffer) the nucleic acid manipulation reagents are taken up by the cell.

Upon release from capsules, the nucleic acid manipulation reagents can be taken up by the single cells using any suitable method. For example, the cells may undergo electroporation, where the electroporated cells are able to take up the reagents for altering gene product expression in the presence of an electroporation buffer. In another instance, transfection reagents may be used to allow for transfection of the reagents for altering gene product expression. A viral based system may also be used to introduce the nucleic acid manipulation reagents into the single cells.

Capsules described herein serve as carriers for delivery of suitable reagents for nucleic acid manipulation of target nucleic acids to cells (e.g., single cells) in partitions. Such reagents are useful, for example, for altering expression of gene products. Reagents that alter expression of gene products can alter expression by acting on the coding region of the gene of interest or by acting on a non-coding regulatory region of a gene of interest (e.g., enhancers or promoters). Such reagents may alter expression of a gene product by increasing or decreasing expression of the gene product.

The reagents used in the subject methods may allow for the high throughput manipulation of one particular target nucleic acid (i.e., DNA or RNA) or for the high throughput alteration of the expression of a plurality of different target nucleic acids in a single cell. In instances where alteration of a plurality of different target nucleic acids is desired, a plurality of different target nucleic acids may be altered within a single cell or a single target nucleic acid may be altered within a single cell, depending on the reagents included with each capsule. For example, the plurality of capsules used with the subject methods may contain about 100-10,000 different reagents for altering expression of at least one gene product, such that different individual cells receive different reagents.

Any suitable reagents for the manipulation of target nucleic acids can be used with the systems and methods provided herein. Exemplary reagents include, but are not limited to, zinc finger nucleases; Transcription Activator-Like Effector Nucleases (TALENs); reengineered homing nucleases; RNA interference (RNAi) reagents and Clustered Regularly Interspaced Short Palindromic Repeats (CRISPR)/Cas nuclease systems.

In some instances, the nucleic acid manipulation reagents used with the subject methods and systems include CRISPR system reagents. Such reagents include, for example, a nucleic acid encoding a nuclease, such as a RNA-guided nuclease (e.g., Cas9 nuclease or a Cpf1 nuclease) and a nucleic acid encoding a guide RNA (gRNA). Exemplary CRISPR system reagents and methods for use in the subject methods and systems are described in further detail herein and are also known in the art, for example in Shalem et al., Nature Reviews Genetics 16: 299-311 (2013); Zhang et al., Human Molecular Genetics 23(R1): R40-6 (2014); and Zhu et al. Cell 157: 1262-1278 (2014), which are herein incorporated by reference in their entirety for all purposes, and particularly for all teachings relating to CRISPR system reagents.

In an exemplary CRISPR system, a gRNA/RNA-guided nuclease complex is recruited to a genomic target sequence by the base-pairing between the gRNA sequence and the complement to the genomic target sequence. For successful binding of a RNA-guided nuclease, the genomic target sequence must generally also contain the correct Protospacer Adjacent Motif (PAM) sequence immediately following the target sequence (learn more about PAM sequences). The binding of the gRNA/RNA-guided nuclease complex localizes the RNA-guided nuclease to the genomic target sequence so that the RNA-guided nuclease can cut both strands of DNA at the target sequence, causing a Double Strand Break (DSB). RNA-guided nucleases that can be used with the methods and systems provided herein include, but are not limited to, Cas9 nucleases and Cpf1 nucleases.

This DSB can subsequently be repaired through either (1) the Non-Homologous End Joining (NHEJ) DNA repair pathway or (2) the Homology Directed Repair (HDR) pathway. The NHEJ repair pathway often results in inserts/deletions (InDels) at the DSB site that can lead to frameshifts and/or premature stop codons, effectively disrupting the open reading frame (ORF) of the targeted gene. Such types of genomic alterations are useful, for example, for loss-of-function gene function studies. The HDR pathway requires the presence of a repair template, which is used to fix the DSB. Specific nucleotide changes can be introduced into a targeted gene by the use of HDR with a repair nucleic acid template. The HDR pathway can be used, for example, to introduce gain of function mutations or to modify regulatory elements.

The RNA-guided nuclease used with the subject systems and methods depends on the particular type of gene alteration desired. For example, the RNA-guided nuclease may be an inducible RNA-guided nuclease (e.g., Cas9 or Cpf1) that is optimized for expression in a temporal or cell-type dependent manner. Mutant Cas9 nucleases that exhibit improve specificity may also be used (see, e.g., Ann Ran et al. Cell 154(6) 1380-89 (2013), which is herein incorporated by reference in its entirety for all purposes, and particularly for all teachings relating to mutant Cas nucleases). Further, deactivated RNA-guided nucleases (i.e., nuclease-null) may be used as a homing device for other proteins (e.g., transcriptional repressor or activators) that affect gene expression at the target site.

Guide RNAs (gRNAs) used with the subject systems and methods may target coding regions or regulatory non-coding regions (e.g., enhancers and promoters). The number and types of gRNAs used depend on the application of the systems and methods described herein. For example, the systems and methods may be used for large scale mutagenesis that employ a guide RNA library containing a plurality of guide RNAs that targets a plurality of different target sequences. The systems and methods may also be used to introduce one particular alteration using one specific gRNA in a large number of one particular cell type or many different types of cells. For example, a particular gRNA may be used to correct a disease loss of function gene or to inactivate a disease gene associated with a dominant-negative disorder.

In applications where introduction of a specific allele or mutation is desired, the nucleic acid manipulation reagents also include a homology repair template nucleic acid that includes the specific allele mutation. The homology repair template nucleic acid introduces the specific allele mutation into the genome of a cell upon repair of a Cas induced DSB through the HDR pathway. In some instances, the homology repair template is used to introduce a specific mutation into a wild type cell. In other instances, the homology repair template is used to introduce a wild type allele into a mutant cell (e.g., a cell containing a mutation associated with a particular disease). The homology repair template may further include a label for identification and sorting of cells containing the specific mutation, for example, a nucleic acid or fluorescent barcode label as described herein. In such applications where introduction of a specific mutation via a homology repair template nucleic acid is desired, the reagents may also include one or more reagents that promote the HDR pathway over HNEJ repair of DSBs. Such reagents include, but are not limited to agents that repress genes involved in HNEJ repair, for example, DNA ligase IV (see, e.g., Maruyana et al. Nat Biotechnol. 33(5): 538-42 (2015), which is herein incorporated by reference in its entirety for all purposes, and particularly for all teachings relating to agents that repress genes involved in HNEJ repair.

The subject capsules can function as carriers for the nucleic acid manipulation reagents in a variety of different ways. For instance, the reagents can be encapsulated with the capsules. Such capsules may have an outer barrier surrounding an inner fluid center or core, for example, a droplet in an emulsion. In other instances, capsules may include a cross-linked polymer or a porous matrix that is capable of entraining and/or retaining materials within its matrix. Capsules used with the subject systems and methods may also include a bead (e.g., a gel bead), where the reagents described herein are attached to the beads.

Capsules used with the methods and systems provided herein are configured to release their contents (e.g., reagents) upon the application of a stimulus after the capsules are delivered or separated into discrete partitions containing individual cells. Individual capsules may contain reagents for the alteration of expression of one gene product (e.g., one guide RNA) or more than one gene product (e.g., more than one guide RNA). In addition, the subject capsules may also contain other reagents that facilitate the delivery of the nucleic acid manipulation reagents into the cells, for example, transfection reagents.

Each capsule may further include a label that allows for the identification and/or sorting of the capsule. Such labels are useful, for example, for partitioning or introducing capsules containing reagents for the editing of particular target sequences with particular cell types, for example, in applications where a plurality of different reagents and/or cell types are used. Suitable labels include, for example, fluorescent labels and unique nucleic acid barcodes described herein.

According to the subject methods, capsules containing the reagents described herein are “delivered” or “separated into” discrete partitions containing individual cells. As used herein, “delivered” and “separated into” are used interchangeably to describe the process by which capsules containing reagents are introduced into partitions containing cells for which altering gene expression is desired.

Any suitable cells can be used with the subject methods and systems described herein. Exemplary cells include, but are not limited to, bacteria, plant, yeast and mammalian cells, including, human cells. Depending on the application of the subject methods, either a single cell type or multiple cell types may be used. In certain instances, the cells (e.g., stem cells) are used for the manufacture of cell based therapies. In other instances, fertilized embryos at the single cell stage may be used for creating transgenic animals.

In some aspects, the compartments or partitions containing the cells that undergo nucleic acid manipulation include partitions that are flowable within fluid streams. These partitions may comprise, for example, microcapsules or micro-vesicles that have an outer barrier surrounding an inner fluid center or core, or they may be a porous matrix that is capable of entraining and/or retaining materials within its matrix. In some aspects, however, these partitions comprise droplets of aqueous fluid within a non-aqueous continuous phase, for example, an oil phase. A variety of different vessels are described in, for example, U.S. Patent Publication No. 2014/0155295, the full disclosure of which is incorporated herein by reference in its entirety for all purposes, and in particular for all teachings related to partitions and droplets used in accordance with the present invention. Likewise, emulsion systems for creating stable droplets in non-aqueous or oil continuous phases are described in detail in, e.g., U.S. Patent Publication No. 2010/0105112.

In the case of droplets in an emulsion, allocating individual cells to discrete partitions may generally be accomplished by introducing a flowing stream of cells in an aqueous fluid into a flowing stream of a non-aqueous fluid, such that droplets are generated at the junction of the two streams. By providing the aqueous cell-containing stream at a certain concentration level of cells, one can control the level of occupancy of the resulting partitions in terms of numbers of cells. In some cases, where single cell partitions are desired, it may be desirable to control the relative flow rates of the fluids such that, on average, the partitions contain less than one cell per partition, in order to ensure that those partitions that are occupied, are primarily singly occupied. Likewise, one may wish to control the flow rate to provide that a higher percentage of partitions are occupied, e.g., allowing for only a small percentage of unoccupied partitions. In some aspects, the flows and channel architectures are controlled as to ensure a desired number of singly occupied partitions, less than a certain level of unoccupied partitions and less than a certain level of multiply occupied partitions. In many cases, the systems and methods are used to ensure that the substantial majority of occupied partitions (partitions containing one or more capsules) include no more than 1 cell per occupied partition. In some cases, the partitioning process is controlled such that fewer than 25% of the occupied partitions contain more than one cell, and in many cases, fewer than 20% of the occupied partitions have more than one cell, while in some cases, fewer than 10% or even fewer than 5% of the occupied partitions include more than one cell per partition.

In certain cases, microfluidic channel networks are particularly suited for generating partitions as described herein. Examples of such microfluidic devices include those described in detail in Provisional U.S. Patent Application No. 61/977,804, filed Apr. 4, 2014, the full disclosure of which is herein incorporated by reference in its entirety for all purposes, and particularly for all teachings relating to microfluidic devices. Alternative mechanisms may also be employed in the partitioning of individual cells, including porous membranes through which aqueous mixtures of cells are extruded into non-aqueous fluids. Such systems are generally available from, e.g., Nanomi, Inc.

In the case of droplets in an emulsion, allocating individual cells to discrete partitions may generally be accomplished by introducing a flowing stream of cells in an aqueous fluid into a flowing stream of a non-aqueous fluid, such that droplets are generated at the junction of the two streams. By providing the aqueous cell-containing stream at a certain concentration level of cells, one can control the level of occupancy of the resulting partitions in terms of numbers of cells. In some cases, where single cell partitions are desired, it may be desirable to control the relative flow rates of the fluids such that, on average, the partitions contain less than one cell per partition, in order to ensure that those partitions that are occupied, are primarily singly occupied. Likewise, one may wish to control the flow rate to provide that a higher percentage of partitions are occupied, e.g., allowing for only a small percentage of unoccupied partitions. In some aspects, the flows and channel architectures are controlled as to ensure a desired number of singly occupied partitions, less than a certain level of unoccupied partitions and less than a certain level of multiply occupied partitions.

Each cell containing partition may also include an identification label that advantageously allows for the identification, tracking and sorting of particular cells and/or reagents. For example, in systems and methods that employ a CRISPR system and a plurality of cell types, such identification labels may allow for certain guide RNAs to be sorted and partitioned with specific cell-types in the plurality of different cell types. Suitable labels may include, for example, fluorescent labels and nucleic acid barcode labels described herein. Such barcodes may also facilitate the identification of particular mutations associated with particular phenotypes, for example, in a mutagenesis screen.

After capsules containing the reagents are separated or delivered into partitions containing an individual cell, the capsules are caused to release their contents into the partitions under conditions that enable uptake of the reagents by the cells. In some instances, the capsules are caused to release their contents using a stimulus delivered to the capsule. Any suitable stimulus may be used to cause the capsules to release their contents. Exemplary stimuli include, but are not limited, a chemical stimulus, an electrical stimulus, a thermal stimulus, a magnetic stimulus, a change in pH, a change in ion concentration, reduction of disulfide bonds, and a photo-stimulus.

Nucleic acid manipulation reagents are taken up by cells once the reagents are released from capsules. Uptake by cells can be facilitated by the inclusion of uptake reagents in the partition, for example, transfection reagents or electroporation buffers.

Individual capsules containing cells that have undergone a nucleic acid manipulation event may then be further sorted and analyzed depending on the application. For example, in a phenotypic screen, such cells may be placed under selective conditions for a particular phenotype and cells having the particular phenotype can be characterized using any suitable techniques including, for example, fluorescence, luminescence and high-content imaging techniques. See, e.g., Hasson et al., Nature 504: 291-295 (2013); Neumann et al., Nature Methods 3: 385-390 (2006); and Moffat et al., Cell 124: 1283-1298 (2006). The nucleic acid manipulation in cells that have been selected for a particular phenotype may also be analyzed using suitable sequencing techniques, including, for example, next-generation sequencing techniques.

In some instances, individual cells that have been selected for a particular phenotype are lysed. Such lysis can occur within partitions containing the individual cells with the particular phenotype or cells containing the same phenotype can be combined prior to lysis. Following lysis, reverse transcription of mRNA from the selected cells may be performed in a partition described herein to produce single cell transcriptome profiles. In instances where the individual cells are lysed within partitions, reagents for reverse transcription can be subsequently introduced into each partition. Following reverse transcription, cDNA transcripts are sequenced to identify particular transcripts that are differentially expressed in a particular cell over time, or after exposure to a particular condition as compared to cells that do not exhibit the desired phenotype. Such differential expression is suggestive of genes that contribute to the particular phenotype. See, e.g., US Patent Application Publication No. 2014/0227684, the full disclosures of which is herein incorporated by reference in its entirety for all purposes, and particularly for all teachings relating to methods of reverse transcription in partitioned individual cells.

The subject methods and systems provided herein may be used in a variety of applications where high throughput alteration of at least one gene product in a cell is desired. For instance, the subject methods and systems may be used for large scale mutagenesis. Large scale mutagenesis is useful, for example, for drug development, biological pathway studies and gene function studies. The subject methods and systems may be used for mass generation of transgenic plants and animals. The subject methods and systems can also be used for the large scale production of cell-based therapeutics. For example, the subject methods can be used to create T cells with modified chimeric antigen T cell receptors that are useful in treatment of cancers.

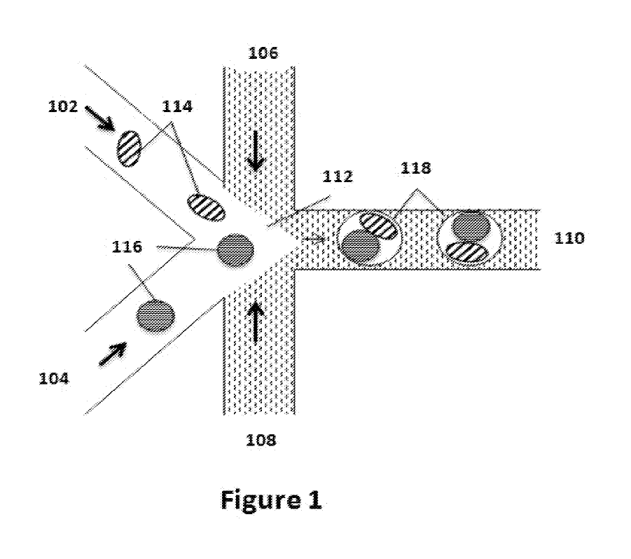

Also provided herein are the microfluidic devices used for delivering reagents (e.g., reagents for altering expression of at least one gene product) as described above. Such microfluidic devices can comprise channel networks for carrying out the delivery process like those set forth in FIG. 1.

### II. WORK FLOW OVERVIEW

In one exemplary aspect, the methods and systems described herein provide for high throughput delivery of reagents in a target cell. In particular, the methods and systems provided herein are used for the delivery of nucleic acid manipulation reagents. Such nucleic acid manipulation reagents can be used, for example, for altering the expression of a gene product encoded by the target nucleic acid. The nucleic acid manipulation reagents can also be used for introducing specific mutations in a particular target nucleic acid, thereby creating mutant gene products that function differently than wildtype counterparts.

In a first step, a plurality of capsules that include the reagents for editing of a target nucleic acid in a target cell is provided. Any suitable reagents for editing of a target nucleic acid can be used with the systems and methods provided herein.

The systems and methods provided herein allow for the high throughput alteration of at least one target nucleic acid in target cells. Target nucleic acids selected for modification may in some instances be sequences with few or no close relatives within the target cell genome. Different target nucleic acids may be located within the same gene or on different genes. In addition, target nucleic acids may encompass whole genes or parts of genes. In some instances, the subject methods provided herein are for the high throughput nucleic acid manipulation of a plurality of target cells, wherein one target nucleic acid is manipulated in each cell. In certain instances, the subject methods provided herein are used for the manipulation of a plurality of target cells, wherein the expression of one gene product is manipulated per single cell. In such embodiments, where the subject method is for the manipulation of the expression of one gene product per single cell, more than one target nucleic acid in the gene encoding the gene product may be manipulated in each single cell. For example, each single cell may be partitioned with nucleic acid manipulation reagents that target 2, 3, 4, 5, 6, 7, 8, 9, 10, 11, 12, 13, 14, 15, 16, 17, 18, 19, 20 or more different target nucleic acids within the same gene.

In other instances, the methods described herein are used for the high throughput alteration of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 200, 300, 400, 500, 600, 700, 800, 900, or 1,000 or more different target nucleic acids in a single cell. In some instances, the methods described herein are used for the high throughput alteration of 2 to 10, 15 to 25, 20 to 30, 35 to 40, 45 to 55, 50 to 60, 65 to 75, 70 to 80, 75 to 85, 80 to 90, 85 to 95, or 90 to 100 different target nucleic acids in a single cell.

In certain instances, the methods described herein are used for the high throughput alteration of at least 50, 100, 200, 300, 400, 500, 600, 700, 800, 900, 1,000, 2,000, 3,000, 4,000, 5,000, 6,000, 7,000, 8,000, 9,000, 10,000, 20,000, 30,000, 40,000, 50,000, 60,000, 70,000, 80,000, 90,000, 1000,000, 2000,000, 300,000, 400,000, 500,000, 600,000, 700,000, 800,000, 900,000, 1 million, 1.5 million, 2 million, 3 million, 4 million, 5 million, 6 million, 7 million, 8 million, 9 million, or 10 million or more single cells. In certain embodiments, the methods are for the high throughput alteration of 50 to 1,000, 1,000 to 5,000, 5,000 to 10,000, 10,000 to 50,000, 50,000 to 100,000, 100,00 to 200,000, 200,000 to 300,000, 300,000 to 400,000, 400,000 to 500,000, 500,000 to 1 million, 1 million to 2 million, 2 million to 3 million, 3 million to 4 million, 4 million to 5 million, 5 million to 6 million, 6 million to 7 million, 7 million to 8 million, 8 million to 9 million, or 9 million to 10 million single cells or more.

The nucleic acid manipulation reagents can act on DNA (e.g., CRISPR system reagents) and/or RNA (e.g., RNAi reagents) nucleic acid targets. The nucleic acid manipulation reagents described herein can be used for altering a target nucleic acid in a manner such that the expression of one or more gene products encoded by the target nucleic acid is altered. For example, in some instances, the nucleic acid manipulation reagents decrease the expression and/or function of one or more gene products. In such instances, the nucleic acid manipulation reagent may target a region that encodes for the gene product or a regulatory region that controls transcription of the nucleic acid. In some instances, the nucleic acid manipulation reagents increase the expression of one more gene products. Nucleic acid manipulation reagents that can be used to increase the expression of a gene product include those that target a regulatory region that affects transcription of the target nucleic acid. In some instances, the nucleic acid manipulation reagent functions by recruiting transcriptional repressors, activators and/or recruitment domains that affect gene expression at the target site without introducing irreversible mutations to the target nucleic acid. In other instances, the nucleic acid manipulation reagent is used for introducing a new mutation into the target gene of interest, such that the mutation confers a new function as compared to the wild type gene product (i.e., a gain-of-function mutation). The nucleic acid manipulation reagents described herein can also be used to introduce a mutation that acts antagonistically to the wild-type version of the gene product (i.e., a dominant-negative mutation).

Suitable reagents nucleic acid manipulation reagents for use with the subject systems and methods provided include, but are not limited to, zinc finger nucleases; Transcription Activator-Like Effector Nucleases (TALENs); reengineered homing nucleases; RNAi reagents such as small interfering RNAs (siRNAs) and small hairpin RNAs (shRNAs); and Clustered Regularly Interspaced Short Palindromic Repeats (CRISPR)/RNA-guided nuclease systems. Nucleic acid manipulation reagents can be delivered to the partitions of single cells, for example, in the form of expression plasmids encoding the reagents, mRNA encoding reagents or viral vectors encoding the reagents.

In some instances, the target nucleic acid manipulation reagents used with the subject methods and systems include CRISPR system reagents. Such reagents include, for example, a nucleic acid encoding a RNA-guided nuclease (e.g., Cas9 nuclease or a Cpf1 nuclease) and a nucleic acid encoding a guide RNA (gRNA), which includes a CRISPR RNA (crRNA) in combination with a trans-activating CRISPR RNA (tracrRNA). The nucleic acids encoding the RNA-guided nuclease and guide RNA may each by operably linked to a regulatory element and may be included on a single vector or on different vectors. Vectors selected may be capable of stable integration into a cellular genome. In some examples, the RNA-guided nuclease (e.g., Cas9 nuclease or a Cpf1 nuclease) and guide RNA do not occur in nature together. Exemplary CRISPR system reagents and methods of use in the present invention are described in further detail herein, for example in Shalem et al., Nature Reviews Genetics 16: 299-311 (2013); Zhang et al., Human Molecular Genetics 23(R1): R40-6 (2014); Zetche et al., http://dx.doi.org/10.1016/j.ce11.2015.09.038, and Zhu et al. Cell 157: 1262-1278 (2014), which are herein incorporated by reference in its entirety for all purposes, and particularly for all teachings relating to CRISPR system reagents.

In the CRISPR system, a gRNA/RNA-guided nuclease complex is recruited to a genomic target sequence by the base-pairing between the gRNA sequence and the complement to the target nucleic acid. The binding of the gRNA/RNA-guided nuclease complex localizes the RNA-guided nuclease (e.g., Cas9 nuclease or a Cpf1 nuclease) to the genomic target sequence so that the wild-type nuclease can cut both strands of DNA causing a Double Strand Break (DSB).

The DSB can be repaired through either (1) the Non-Homologous End Joining (NHEJ) DNA repair pathway or (2) the Homology Directed Repair (HDR) pathway. The NHEJ repair pathway often results in inserts/deletions (InDels) at the DSB site that can lead to frameshifts and/or premature stop codons, effectively disrupting the open reading frame (ORF) of the target nucleic acid, thereby decreasing the expression of the gene product encoded by the target nucleic acid. Such gene alterations are useful, for example, for gene function studies. The HDR pathway requires the presence of a repair template, which is used to fix the DSB. Specific nucleotide changes can be introduced into a targeted gene by the use of HDR with a repair template. The HDR pathway can be used, for example, to introduce gain of function mutations or particular point mutations into the target single cell. RNA-guided nuclease used with the subject methods provided herein can include any suitable nuclease compatible with CRIPSR systems. Suitable nucleases include, but are not limited to, CasI, CasIB, Cas2, Cas3, Cas4, Cas5, Cas6, Cas7, Cas8, Cas9, CasIO, CbfI, CsyI, Csy2, Csy3, CseI, Cse2, CscI, Csc2, Csa5, Csn2, Csm2, Csm3, Csm4, Csm5, Csm6, CmrI, Cmr3, Cmr4, Cmr5, Cmr6, CpfI, Csb I, Csb2, Csb3, CsxI7, CsxI4, CsxIO, CsxI6, CsaX, Csx3, CsxI, CsxI 5, CsfI, Csf2, Csf3, Csf4, C2cI, C2c2, C2c3, homologs thereof, and modified versions thereof.

The RNA-guided nuclease used with the subject systems and methods depend on the particular type of gene manipulation desired. For example, the RNA-guided nuclease may be an inducible RNA-guided nuclease that is optimized for expression in a temporal or cell-type dependent manner. Suitable inducible promoters that can be linked to the RNA-guided nuclease include, but are not limited to light (e.g., green-light or blue-light inducible promoters), heat (e.g., HSP promoters) and chemically inducible promoters (e.g., antibiotic, copper, alcohol, and steroid inducible promoters). See, e.g., Papatriantafyllou et al., Nature Reviews Molecular Cell Biology 13, 210 (2012); Yu et al., Protist 163(2):284-95 (2012); and Lee et al., Appl Environ Microbiol 76(10): 3089-3096 (2010), which are herein incorporated by reference in entirety for all purposes, and particularly for all teachings relating to inducible promoters. Exemplary promoters include, for example, tetracycline-inducible promoters, metallothionein promoters; tetracycline-inducible promoters, methionine-inducible promoters (e.g., MET25, MET3 promoters); and galactose-inducible promoters (GAL1, GAL7 and GAL 10 promoters). Other suitable promoters include the ADH1 and ADH2 alcohol dehydrogenase promoters (repressed in glucose, induced when glucose is exhausted and ethanol is made), the CUP1 metallothionein promoter (induced in the presence of Cu²⁺, Zn²⁺), the PHO5 promoter, the CYC1 promoter, the HIS3 promoter, the PGK promoter, the GAPDH promoter, the ADC1 promoter, the TRP1 promoter, the URA3 promoter, the LEU2 promoter, the ENO promoter, the TP1 promoter, and the AOX1 promoter.

Mutant RNA-guided nucleases that exhibit improve specificity may also be used (see, e.g., Ann Ran et al. Cell 154(6) 1380-89 (2013), which is herein incorporated by reference in its entirety for all purposes, and particularly for all teachings relating to mutant RNA-guided nucleases with improved specificity for target nucleic acids). The nucleic acid manipulation reagents can also include deactivated RNA-guided nucleases (e.g., Cas9 (dCas9)). Deactivated RNA-guided nucleases provided herein can be used in applications in which cutting at a particular target nucleic acid is not desired. Deactivated Cas9 binding to nucleic acid elements alone may repress transcription by sterically hindering RNA polymerase machinery and stalling transcription elongation. Further, deactivated Cas may be used as a homing device for other proteins (e.g., transcriptional repressor, activators and recruitment domains) that affect gene expression at the target site without introducing irreversible mutations to the target nucleic acid. For example, dCas9 can be fused to transcription repressor domains such as KRAB or SID effectors to promote epigenetic silencing at a target site. Cas9 can also be converted into a synthetic transcriptional activator by fusion to VP16/VP64 or p64 activation domains.

Such deactivated RNA-guided nucleases (e.g., dCas9) can also be used as a homing device for epigenetic modification tools. Deactivated RNA-guided nucleases fused to epigenetic modification tools can be used for the modification of histone tails and DNA molecules, such as histone methylation and demethylation, histone acetylation, cytosine methylation and hydroxymethylation. For example, a deactivated RNA-guided nuclease may be fused to the functional domain of a DNA methyltransferase for targeted CpG promoter site methylation. A deactivated nuclease can be fused to an epigenetic modification tool for removal of the methylation from key promoter CpGs (e.g., the hydrocylase catalytic domain of TET1). See, e.g., Falahi et al., Mol. Cancer Res. 11: 1029-1039 (2013); Mendenhall et al., Nat. Biotechnol. 31: 1133-1136 (2013); and Hilton et al., Nat. Biotechnol. 33: 510-517 (2015), which is herein incorporated by reference in its entirety for all purposes, and particularly for all teachings relating to epigenetic modification tools.

In certain instances wherein CRISPR system reagents are used, the guide RNAs are attached to bead capsules (e.g., gel beads) and nucleic acids encoding the RNA-guided nuclease are carried in droplets. In such instances, the guide RNA and nuclease may be partitioned together prior to partitioning with a target cell. Alternatively, the guide RNA and RNA-guided nuclease can each be partitioned directly with the target cell. In some embodiments, the guide RNA and RNA-guided nuclease are partitioned together prior to partitioning with the target cell. In certain embodiments, the guide RNA is partitioned with the target cell prior to the partitioning of the RNA-guided nuclease with the target cell. In other embodiments, the RNA-guided nuclease is partitioned with the target cell prior to the partitioning of the guide RNA with the target cell. In some instances of the subject methods, the guide RNAs and RNA-guided nuclease are each delivered to the target cells using bead capsules.

Guide RNAs (gRNAs) used with the subject systems and methods may target nucleic acid coding regions or regulatory non-coding regions (e.g., enhancers and promoters). The number and types of gRNAs used depend on the application of the systems and methods described herein. For example, the systems and methods may be used for large scale mutagenesis that employ a guide RNA library containing a plurality of gRNAs that targets a plurality of different target sequences. The systems and methods may also be used to introduce one particular alteration using a specific gRNA in a large number of one particular cell type or many different types of cells. For example, a particular gRNA may be used to correct a disease loss of function gene or to inactivate a disease gene associated with a dominant-negative disorder. In some instances, only one particular guide RNA is used. In some instances at least 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, or 1,000 different guide RNAs are used, each different guide RNA corresponding to a different target nucleic acid for alteration. In some instances, 2 to 50, 50 to 100, 100 to 150, 150 to 200, 200 to 250, 250 to 300, 300 to 350, 350 to 400, 450 to 500, 500 to 550, 550 to 600, 600 to 650, 650 to 700, 700 to 750, 750 to 800, 850 to 900, 900 to 950 and 950 to 1,000 different guide RNAs are used. In yet other instances, at least 2,000, 3,000, 4,000, 5,000, 6,000, 7,000, 8,000, 9,000 or 10,000 different guide RNAs are used. In some instances 2,000 to 3,000, 2,500 to 3,000, 3,000 to 4,000, 3,500 to 4,500, 4,000 to 5,000, 4,500 to 5,500, 5,000 to 6,000, 5,500 to 6,500, 6,000 to 7,000, 6,500 to 7,500, 7,000 to 8,000, 7,500 to 8,500, 8,000 to 9,000, 8,500 to 9,500 or 9,000 to 10,000 different guide RNAs are used. In instances where more than one guide RNA is used, each different guide RNA may be associated with a different barcode label that allows for the identification and sorting of the guide RNA as described below. For example, fluorescent labels may allow for the partitioning of particular gRNAs with particular single cells through fluorescent cell sorting techniques. Such barcodes may be included as part of the guide RNA or included as part of the capsules carrying the guide RNA as described below. In some instances, wherein a cell is partitioned with more than one guide RNAs, all of the guide RNAs that are to be partitioned with a particular cell contain the same bar code. In such instances, guide RNAs that are partitioned with different cells contain different bar codes. Such configurations advantageously allow for the sorting and partitioning of guide RNAs with particular cells. Further, such configurations may also advantageously allow the tracking and identification of cells containing particular nucleic acid manipulations following a nucleic acid manipulation event.

Repair of a double strand break created by the RNA-guided nuclease may be repaired by either the error-prone non-homologous end joining (NHEJ) or homology directed repair (HDR). NHEJ typically generates small insertions or deletions (inDels) that are unpredictable in nature, but frequently cause impactful and inactivating mutation in the target nucleic acid. Conversely, the HDR pathway is useful for precise insertion of donor DNA into the target nucleic acid.

In applications where introduction of a specific allele (e.g., a wild type allele to replace a mutant allele or a mutant allele to replace a wild type allele) is desired using a CRISPR system, the nucleic acid manipulation reagents may also include a homology repair template nucleic acid that includes the specific allele. The homology repair template nucleic acid introduces the specific allele into the genome of the target cell upon repair of a RNA-guided nuclease induced DSB through the HDR pathway. The homology repair template may further include a label for identification and sorting of cells containing the specific mutation or allele. In such applications where introduction of a specific allele via a homology repair template nucleic acid is desired, the reagents may also include one or more reagents that promote the HDR pathway over HNEJ repair of DSBs. Such reagents include, but are not limited to agents that repress genes involved in HNEJ repair, for example, DNA ligase IV. See, e.g., Maruyana et al. Nat Biotechnol. 33(5): 538-42 (2015), which is herein incorporated by reference in its entirety for all purposes, and particularly for all teachings relating to agents that repress genes invovled in HNEJ repair of DSBs.

Other exemplary nucleic acid manipulation reagents that can be used with the systems and methods provided herein include reagents that silence the expression of one or more target genes through the RNA interference (RNAi) pathway, including, but are not limited to, small hairpin RNAs (shRNAs), double-stranded RNA (dsRNA), small interfering RNAs (siRNAs), and shRNAs embedded in microRNA (miRNA) precursors (shRNAmirs).

In some instances, the nucleic acid manipulation reagent is used to introduce a detectable label in a target nucleic acid. The number of detectable labels included depends on the application of the method. Detectable labels may be included, for example, in a homology repair template nucleic acid as discussed above. In some instances, the detectable label is used to monitor a particular nucleic acid alteration introduced into the genome of a target cell. In such instances, different detectable labels may be used to differentiate between the different alterations (e.g., different fluorophores or nucleic acid sequences). In some instances, there may be more than one reagent that targets a particular target nucleic acid (e.g., a plurality of “tiling” gRNAs that target overlapping regions of a particular gene). In such a case, the same detectable label may be used for all the gRNAs that target the same gene. Detectable labels include labels that allow for the non-invasive detection of a particular nucleic acid alteration in a cell. When used as a large scale screen, for example, such detectable labels can advantageously allow for the identification of a nucleic acid manipulation that is associated with a particular phenotype of interest.

Detection of the detectable label can be carried out by any suitable method, including fluorescence spectroscopy or by other optical means. In certain cases, the detectable label is a fluorophore, which, after absorption of energy, emits radiation at a defined wavelength. Fluorescent detectable labels include, for example, dansyl-functionalised fluorescent moieties (see, e.g., Welch et al., Chem. Eur. J. 5(3):951-960 (1999)); fluorescent labels Cy3 and Cy5 (see, e.g., Zhu et al., Cytometry 28:206-211 (1997)). Suitable detectable labels are also disclosed in Prober et al., Science 238:336-341 (1987); Connell et al., BioTechniques 5(4):342-384 (1987); Ansorge et al., Nucl. Acids Res. 15(11):4593-4602 (1987) and Smith et al., Nature 321:674 (1986). Other commercially available fluorescent labels include, but are not limited to, fluorescein, rhodamine (including TMR, texas red and Rox), alexa, bodipy, acridine, coumarin, pyrene, benzanthracene and the cyanins.

The nucleic acid manipulation reagents described herein are carried by capsules (e.g., microcapsules) to partitions containing the target cells. As used herein, a capsule includes any suitable container or solid substrate for carrying one or more nucleic acid manipulation reagents. A capsule includes, but is not limited to, a well, a microwell, a hole, a droplet (e.g., a droplet in an emulsion) a spot, and a bead. In some instance, the capsule includes an outer barrier surrounding an inner fluid center or core, for example, a droplet in an emulsion. In other instances, a capsule may include a cross-linked polymer or a porous matrix that is capable of entraining and/or retaining materials within its matrix. In some cases, the capsule is a bead. Suitable beads include, for example, gel beads, paraffin beads, and wax beads. In some cases, the capsule is a gel bead. In some instances where the capsule is a bead, the nucleic acid manipulation reagent is releasably coupled to the capsule. For example, in some cases, a nucleic acid manipulation reagent such as a shRNA, siRNA, or guide RNA oligonucleotide is attached to a bead. In some instances, the capsule is a droplet where the nucleic acid manipulation reagent is encapsulated in the droplet.

Nucleic acid manipulation reagents can be coupled to or immobilized on bead capsules using any suitable method. For instance, coupling/immobilization may be via any form of chemical bonding (e.g., covalent bond, ionic bond) or physical phenomena (e.g., Van der Waals forces, dipole-dipole interactions, etc.). In some cases, coupling/immobilization of a nucleic acid manipulation reagent to a gel bead or any other capsule described herein may be reversible, such as, for example, via a labile moiety (e.g., via a chemical cross-linker, including chemical cross-linkers described herein). Upon application of a stimulus, the labile moiety may be cleaved and the immobilized reagent set free. In some cases, the labile moiety is a disulfide bond. For example, in the case where a nucleic acid manipulation reagent (e.g., a guide RNA) is immobilized to a gel bead via a disulfide bond, exposure of the disulfide bond to a reducing agent can cleave the disulfide bond and free the nucleic acid manipulation reagent from the bead.

In some examples, all of the nucleic acid manipulation reagents for alteration of one or more specific target nucleic acids (i.e., a nucleic acid manipulation reagent “set”) are carried in the same capsule. For example, in instances where a CRISPR system is used in conjunction with bead capsules, an oligonucleotide encoding a CRISPR guide RNA specific for a particular target nucleic acid and an oligonucleotide encoding a RNA-guided nuclease may be releasably attached to the same bead. In some instances, more than one nucleic acid manipulation reagent may be used to alter the expression of a product encoded by a target nucleic acid. For example, a “tiling” method may be used that includes a plurality of reagents that target overlapping regions over the length of a target nucleic acid (e.g., a plurality of gRNAs that target different sequence in a target nucleic acid). In such example, all of the different reagents that target different regions of one particular target nucleic may carried by the same capsule.

In some instances, each component of a set of nucleic acid manipulation reagents (e.g., a CRISPR guide RNA and a RNA-guided nuclease oligonucleotide) is carried by different capsules. In such methods and systems, the individual capsules carrying the different components are each introduced or separated into the same partition containing the target cell, thereby partitioning the target with a full “set” of nucleic acid manipulation reagents with the target cell to allow for the manipulation of a particular target nucleic acid. In certain embodiments, the guide RNAs are carried by beads (e.g., gel beads) and the RNA-guided nuclease is carried using a droplet capsule.

The capsules of the subject systems and methods provided herein may also include a label to allow for the identification, segregation and separation of the capsules in one or more steps of the method. Labels include, but are not limited to fluorescent labels and oligonucleotide “barcodes”. In cases where an oligonucleotide barcode is used, the barcode may be included on the same oligonucleotide as an oligonucleotide encoding one or more reagents for altering gene product expression (e.g., an shRNA, a siRNA or a gRNA) or on a different oligonucleotide. In instances where bead capsules are used, the barcodes may be directly attached to a capsule. Barcodes that are attached to a capsule (e.g., a bead) may be releasably attached. Each bead may typically be provided with large numbers of oligonucleotide molecules attached. In particular, the number of molecules of barcodes on an individual bead may be at least about 10,000 barcode molecules, at least 100,000 barcode molecules, at least 1,000,000 barcode molecules, at least 100,000,000 barcode molecules, and in some cases at least 1 billion barcode molecules.

Reagents and labels may be releasable from a capsule (e.g., a bead) upon the application of a particular stimulus to the capsule. In some cases, the stimulus may be a photo-stimulus, e.g., through cleavage of a photo-labile linkage that may release the oligonucleotides. In some cases, a thermal stimulus may be used, where elevation of the temperature of the beads environment may result in cleavage of a linkage or other release of the oligonucleotides form the beads. In some cases, a chemical stimulus may be used that cleaves a linkage of the oligonucleotides to the beads, or otherwise may result in release of the oligonucleotides from the beads. Examples of this type of system are described in U.S. Patent Publication No. 2014/0155295, as well as U.S. Provisional Patent Application Nos. 61/940,318, filed Feb. 7, 2014, 61/991,018, Filed May 9, 2014, and U.S. Patent Publication No. 2014/0378345, the full disclosures of which is herein incorporated by reference in its entirety for all purposes, and particularly for all teachings relating to methods of releasably attaching oligonucleotides to beads. In one case, such compositions include the polyacrylamide matrices described above for encapsulation of cells, and may be degraded for release of the attached oligonucleotides through exposure to a reducing agent, such as DTT. In some cases, the stimulus is applied in a manner and under conditions that causes the capsule to dissolve, thereby releasing the reagents from the capsule.

In accordance with the methods and systems described herein, capsules that include the nucleic acid manipulation reagents are delivered into or segregated into discrete partitions containing individual cells. As used “delivered into” and “segregate into” are used interchangeably to describe the process of creating a partition that includes at least one cell and at least one set of nucleic acid manipulation reagents. As used herein, a “set” of nucleic acid manipulation reagents refers to the nucleic acid manipulation reagents necessary to carry out the editing of a particular target nucleic acid or target nucleic acids in a cell. For example, a set of nucleic acid manipulation reagents in a CRISPR system includes at least a nucleic acid encoding a RNA-guided nuclease and a guide RNA for localizing the RNA-guided nuclease to the desired target nucleic acid. The reagents in a set of nucleic acid manipulation reagents can be included with the same capsule or different capsules. In some cases, nucleic acid manipulation reagents (e.g., shRNA, siRNA, or gRNA) are attached to a bead capsule, wherein the bead is delivered into a partition such that a single bead and a single cell are contained within an individual partition. While single cell/single set of nucleic acid manipulation reagent occupancy is the most desired state, it will be appreciated that multiple occupied partitions (either in terms of cells, beads or both), or unoccupied partitions (either in terms of cells, beads or both) will often be present. In some cases, the ratio of cells to capsules carrying the nucleic acid reagents in a single partition is 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10.

In some instances where CRISPR system nucleic acid manipulation reagents are used, the guide RNA and RNA-guided nuclease components can be included on separate beads. In some cases of such a configuration, the ratio of guide RNA carrying beads to RNA-guided nuclease carrying beads in a particular partition is 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In some instances of such a configuration, the cell, bead carrying the guide RNA and bead carrying the RNA-guided nuclease are present in the partition at a ratio of 1:1:1.

As used herein, the partitions refer to containers or vessels that may include a variety of different forms, e.g., wells, tubes, micro or nanowells, through holes, or the like. In preferred aspects, however, the partitions are flowable within fluid streams. These vessels may be comprised of, e.g., microcapsules or micro-vesicles that have an outer barrier surrounding an inner fluid center or core, or they may be a porous matrix that is capable of entraining and/or retaining materials within its matrix. In some aspects, these partitions may comprise droplets of aqueous fluid within a non-aqueous continuous phase, e.g., an oil phase. A variety of different vessels are described in, for example, U.S. Patent Publication No. 2014/0155295. Likewise, emulsion systems for creating stable droplets in non-aqueous or oil continuous phases are described in detail in, e.g., U.S. Patent Publication No. 2010/0105112. In certain cases, microfluidic channel networks are particularly suited for generating partitions as described herein. Examples of such microfluidic devices include those described in detail in Provisional U.S. Patent Application No. 61/977,804, filed Apr. 4, 2014, the full disclosure of which is herein incorporated by reference in its entirety for all purposes, and particularly for all teachings relating to microfluidic devices. Alternative mechanisms may also be employed in the partitioning of individual cells, including porous membranes through which aqueous mixtures of cells are extruded into non-aqueous fluids. Such systems are generally available from, e.g., Nanomi, Inc.

In the case of droplets in an emulsion, partitioning of cells and capsules carrying nucleic acid manipulation reagents into discrete partitions may generally be accomplished by flowing an aqueous, sample containing stream, into a junction into which is also flowing a non-aqueous stream of partitioning fluid, e.g., a fluorinated oil, such that aqueous droplets are created within the flowing stream partitioning fluid, where such droplets include the sample materials. The relative amount of cells and capsules carrying nucleic acid manipulation reagents within any particular partition may be adjusted by controlling a variety of different parameters of the system, including, for example, the concentration of cells or capsules carrying nucleic acid manipulation reagents in the aqueous stream, the flow rate of the aqueous stream and/or the non-aqueous stream, and the like.

Microfluidic devices may be used to provide for the controlled partitioning of cells and capsules containing nucleic acid reagents. In some instances, microfluidic devices that include a network of microfluidic channel structures are used to deliver the nucleic acid manipulation reagents and cells into the same partition. Examples of such microfluidic devices include those described in detail in Provisional U.S. Patent Application No. 61/977,804, filed Apr. 4, 2014, the full disclosure of which is herein incorporated by reference in its entirety for all purposes, and particularly for all teachings relating to microfluidic devices.

An example of a microfluidic channel structure for co-partitioning cells and beads that include gene expression altering reagent oligonucleotides is schematically illustrated in FIG. 1. As described herein, in some aspects, a substantial percentage of the overall occupied partitions will include both a bead and a cell and, in some cases, some of the partitions that are generated will be unoccupied. In some cases, some of the partitions may have beads and cells that are not partitioned 1:1. In some cases, it may be desirable to provide multiply occupied partitions, e.g., containing two, three, four or more cells and/or beads within a single partition. As shown, channel segments 102, 104, 106, 108 and 110 are provided in fluid communication at channel junction 112. An aqueous stream comprising the individual cells 114, is flowed through channel segment 102 toward channel junction 112. As described above, these cells may be suspended within an aqueous fluid, or may have been pre-encapsulated, prior to the partitioning process.

With reference to FIG. 1, an aqueous stream of cells 114 is flowed through channel segment 102 toward channel junction 112. Concurrently, an aqueous stream comprising the nucleic acid manipulation reagent carrying beads 116, is flowed through channel segment 104 toward channel junction 112. A non-aqueous partitioning fluid 116 is introduced into channel junction 112 from each of side channels 106 and 108, and the combined streams are flowed into outlet channel 110. Within channel junction 112, the two combined aqueous streams from channel segments 102 and 104 are combined, and partitioned into droplets 218, that include co-partitioned cells 114 and beads 116. As noted previously, by controlling the flow characteristics of each of the fluids combining at channel junction 112, as well as controlling the geometry of the channel junction, one can optimize the combination and partitioning to achieve a desired occupancy level of beads, cells or both, within the partitions 118 that are generated.

In some cases, where single cell and/or bead partitions are desired, it may be desirable to control the relative flow rates of the fluids such that, on average, the partitions contain less than one cell and/or bead per partition, in order to ensure that those partitions that are occupied, are primarily singly occupied. Likewise, one may wish to control the flow rate to provide that a higher percentage of partitions are occupied, e.g., allowing for only a small percentage of unoccupied partitions. In preferred aspects, the flows and channel architectures are controlled as to ensure a desired number of singly occupied partitions, less than a certain level of unoccupied partitions and less than a certain level of multiply occupied partitions.

The number of microfluidic channels may depend on the number of different reagents used in a particular application. In some cases where the method is performed with two or more nucleic acid manipulation reagents on separate capsules, each of the two or more nucleic acid manipulation reagents are carried in a separate aqueous stream via separate microfluid channels. Such a configuration allows for control over the amount of each individual reagent that is partitioned with each cell. For example, in cases where a CRISPR system is used, a guide RNA are introduced into a partition in a first aqueous stream via a first channel and a RNA-guided nuclease (e.g., a Cas9 nuclease or Cpf1 nuclease) is introduced into the partition in a second aqueous stream via a second channel. In another example, where a CRISPR system is used, the guide RNAs and RNA-guided nuclease are introduced into a partition by in the same aqueous stream via the same channel. In some instances, the streams carrying each of the two or more nucleic acid manipulation reagents are combined into a partition and a stream carrying a full set of nucleic acid manipulation reagents is delivered to the partition containing the target cell. In other instances, each of the streams carrying one of the two or more nucleic acid manipulation reagents is delivered separately to the partition containing the target cell. While certain aspects of the subject systems and methods are described herein in the context of CRISPR systems, one of skill in the art would recognize that other nucleic acid manipulation reagents, including those nucleic acid manipulation reagents described herein, can also be used in conjunction with the subject systems and methods.

The channel networks, e.g., as described herein, can be fluidly coupled to appropriate fluidic components. For example, the inlet channel segments, e.g., channel segments 102, 104, 106 and 108 are fluidly coupled to appropriate sources of the materials they are to deliver to channel junction 112. For example, channel segment 102 will be fluidly coupled to a source of an aqueous suspension of cells 114 to be analyzed, while channel segment 104 would be fluidly coupled to a source of an aqueous suspension of nucleic acid manipulation reagent carrying beads 116. Channel segments 106 and 108 would then be fluidly connected to one or more sources of the non-aqueous fluid. These sources may include any of a variety of different fluidic components, from simple reservoirs defined in or connected to a body structure of a microfluidic device, to fluid conduits that deliver fluids from off-device sources, manifolds, or the like. Likewise, the outlet channel segment 110 may be fluidly coupled to a receiving vessel or conduit for the partitioned cells. Again, this may be a reservoir defined in the body of a microfluidic device, or it may be a fluidic conduit for delivering the partitioned cells to a subsequent process operation, instrument or component.

In many cases, the systems and methods are used to ensure that the substantial majority of occupied partitions (partitions containing one or more microcapsules) include no more than 1 target cell per occupied partition. In some cases, the partitioning process is controlled such that fewer than 25% of the occupied partitions contain more than one target cell, and in many cases, fewer than 20% of the occupied partitions have more than one target cell, while in some cases, fewer than 10% or even fewer than 5% of the occupied partitions include more than one cell per partition.

Additionally or alternatively, in many cases, it is desirable to avoid the creation of excessive numbers of empty partitions. While this may be accomplished by providing sufficient numbers of target cells into the partitioning zone, the poissonian distribution would expectedly increase the number of partitions that would include multiple cells. As such, in accordance with aspects described herein, the flow of one or more of the cells, or other fluids directed into the partitioning zone are controlled such that, in many cases, no more than 50% of the generated partitions are unoccupied, i.e., including less than 1 target cell, no more than 25% of the generated partitions, no more than 10% of the generated partitions, may be unoccupied. Further, in some aspects, these flows are controlled so as to present non-poissonian distribution of single occupied partitions while providing lower levels of unoccupied partitions. Restated, in some aspects, the above noted ranges of unoccupied partitions can be achieved while still providing any of the single occupancy rates described above. For example, in many cases, the use of the systems and methods described herein creates resulting partitions that have multiple occupancy rates of from less than 25%, less than 20%, less than 15%, less than 10%, and in many cases, less than 5%, while having unoccupied partitions of from less than 50%, less than 40%, less than 30%, less than 20%, less than 10%, and in some cases, less than 5%.

As will be appreciated, the above-described occupancy rates are also applicable to partitions that include both target cells and capsules carrying the nucleic acid manipulation reagents. In particular, in some aspects, a substantial percentage of the overall occupied partitions will include both a capsule and a cell. In particular, it may be desirable to provide that at least 50% of the partitions are occupied by at least one cell and at least set of nucleic acid manipulation reagents, or at least 75% of the partitions may be so occupied, or even at least 80% or at least 90% of the partitions may be so occupied. Further, in those cases where it is desired to provide a single cell and a single set of nucleic acid manipulation reagents within a partition, at least 50% of the partitions can be so occupied, at least 60%, at least 70%, at least 80% or even at least 90% of the partitions can be so occupied.

Although described in terms of providing substantially singly occupied partitions, above, in certain cases, it is desirable to provide multiply occupied partitions, e.g., containing two, three, four or more cells and/or capsules (e.g., beads) that include the set of nucleic acid manipulation reagents within a single partition. Accordingly, as noted above, the flow characteristics of the cell and/or capsule (e.g., bead) containing fluids and partitioning fluids may be controlled to provide for such multiply occupied partitions. In particular, the flow parameters may be controlled to provide a desired occupancy rate at greater than 50% of the partitions, greater than 75%, and in some cases greater than 80%, 90%, 95%, or higher. In particular embodiments, the flow parameters are controlled to provide a desired multiple occupancy rate of a single cell and a set of reagents for nucleic acid manipulation at greater than 50% of the partitions, greater than 75%, and in some cases greater than 80%, 90%, 95%, or higher.

Additionally, in many cases, the capsules within a single partition may include different nucleic acid manipulation reagents associated therewith. For example, in methods and systems that employ a CRISPR system, a first capsule may include a first guide RNA and a second capsule may include an oligonucleotide encoding a RNA-guided nuclease. In some instances where two or more target nucleic acids are edited, a third capsule may include a second guide RNA that targets a different nucleic acid than the first guide RNA. In such cases, it may be advantageous to introduce different capsules into a common channel or droplet generation junction, from different capsules sources, i.e., containing different associated reagents, through different channel inlets into such common channel or droplet generation junction. In such cases, the flow and frequency of the different capsules into the channel or junction may be controlled to provide for the desired ratio of microcapsules from each source, while ensuring the desired pairing or combination of such capsules into a partition with the desired number of cells. In one exemplary embodiment, capsules that include different nucleic acid manipulation reagents are delivered at a 1:1 ratio into the partition containing the cell.

The partitions described herein are often characterized by having extremely small volumes, e.g., less than 10 μL, less than 5 μL, less than 1 μL, less than 900 picoliters (pL), less than 800 pL, less than 700 pL, less than 600 pL, less than 500 pL, less than 400 pL, less than 300 pL, less than 200 pL, less than 100 pL, less than 50 pL, less than 20 pL, less than 10 pL, less than 1 pL, less than 500 nanoliters (nL), or even less than 100 nL, 50 nL, or even less.

For example, in the case of droplet based partitions, the droplets may have overall volumes that are less than 1000 pL, less than 900 pL, less than 800 pL, less than 700 pL, less than 600 pL, less than 500 pL, less than 400 pL, less than 300 pL, less than 200 pL, less than 100 pL, less than 50 pL, less than 20 pL, less than 10 pL, or even less than 1 pL. Where co-partitioned with beads, it will be appreciated that the sample fluid volume, e.g., including co-partitioned cells, within the partitions may be less than 90% of the above described volumes, less than 80%, less than 70%, less than 60%, less than 50%, less than 40%, less than 30%, less than 20%, or even less than 10% the above described volumes.

As is described elsewhere herein, partitioning species may generate a population of partitions. In such cases, any suitable number of partitions can be generated to generate the population of partitions. For example, in a method described herein, a population of partitions may be generated that comprises at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions or at least about 1,000,000,000 partitions. Moreover, the population of partitions may comprise both unoccupied partitions (e.g., empty partitions) and occupied partitions.

In addition to the capsule that includes at least one nucleic acid manipulation reagent, one or more other reagents may also be partitioned with the cell undergoing nucleic acid editing. For example, one or more reagents for assisting the uptake of the at least one nucleic acid manipulation reagent into the cell. In some cases, one or more transfection reagents are partitioned with the cell and nucleic acid manipulation reagents. In cases where electroporation is used for the uptake of nucleic acid manipulation reagents, an electroporation buffer may be included in the partitioned with the cell undergoing the nucleic acid editing. Such additional reagents may be partitioned with the capsule that includes a full set of nucleic acid manipulation reagent to a cell or may be delivered to the cell separately from the capsule that includes the nucleic acid manipulation reagent.

After partitioning of cell and capsule containing the at least one nucleic acid manipulation reagent, the capsules are caused to release the nucleic acid manipulation reagents, thereby enabling the uptake of the reagents by the individual cells. In some cases, the stimulus may be a photostimulus, e.g., through cleavage of a photo-labile linkage that may release the oligonucleotides. In some cases, a thermal stimulus may be used, where elevation of the temperature of the beads environment may result in cleavage of a linkage or other release of the oligonucleotides form the beads. In some cases, a chemical stimulus may be used that cleaves a linkage of the oligonucleotides to the beads, or otherwise may result in release of the oligonucleotides from the beads. In the case of a photo or heat stimulus, the stimulus may be introduced to the partition containing the cell and nucleic acid manipulation reagents by a heat or light source through an opening in a microfluidic channel carrying the partition. Chemical stimuli may be partitioned with the target cell prior to partitioning of the target cell and capsules carrying the nucleic acid manipulation reagents. In this example, the nucleic acid manipulation reagents will be released from capsules only in the presence of the target cell and the chemical stimuli.

Upon release of the nucleic acid manipulation reagents from capsules, uptake of the nucleic acid manipulation reagent by the cell may be carried out using any suitable method. As mentioned herein, one or more cell uptake reagents may be included to assist in the uptake of a nucleic acid manipulation reagent into the cell. In some cases, the one or more cell uptake reagents are transfection reagents, including, for example, polymer based (e.g. DEAE dextran) transfection reagents and cationic liposome-mediated transfection reagents. Electroporation of the cell may also be used to facilitate uptake of the nucleic acid manipulation reagents. By applying an external field, an altered transmembrane potential in a cell is induced, and when the transmembrane potential net value (the sum of the applied and the resting potential difference) is larger than a threshold, transient permeation structures are generated in the membrane and electroporation is achieved. See, e.g., Gehl et al., Acta Physiol. Scand. 177:437-447 (2003). Cells used with the subject systems and methods may be electroporated prior to delivery of nucleic acid manipulation reagents or after the partitioning of the nucleic acid manipulation reagents with the cell. In some instances, an electroporation buffer may be delivered into the partition containing the nucleic acid manipulation reagents and target cell to allow for electroporation of the target cell and uptake of the nucleic acid regents. Nucleic acid manipulation reagents may also be delivered through viral transduction into the target cells. Suitable viral delivery systems include, but are not limited to, adeno-associated virus (AAV) retroviral and lentivirus delivery systems. Such viral delivery systems are particularly useful in instances where the cell is resistant to transfection. In instances that use a viral delivery system, viruses (e.g., adeno-associated viruses (AAV), retroviruses or lentiviruses) that carry the reagents (e.g., nucleotides encoding the reagents) may be encapsulated in capsules that are subsequently delivered to partitions containing cells. The viruses, in turn, introduce the reagents into single cells upon release from the capsules. Methods that use a viral-mediated delivery system may further include a step of preparing viral vectors encoding the nucleic acid manipulation reagents and packaging of the vectors into viral particles. Other method of delivery of nucleic acid reagents include lipofection, nucleofection, microinjection, biolistics, virosomes, liposomes, immunoliposomes, polycation or lipid:nucleic acid conjugates, naked DNA, artificial virions, and agent-enhanced uptake of nucleic acids. See, also Neiwoehner et al., Nucleic Acids Res. 42:1341-1353 (2014), which is herein incorporated by reference in its entirety for all purposes, and particularly for all teachings relating to reagent delivery systems.

Each partition of the subject method can include one particular cell type or different cell types, depending on the application desired. For example, the subject methods may be used for the high throughput genetic screen of one particular cell type or the mass production of cell type containing a particular allele (e.g., the replacement of a mutant allele in a particular gene with a wild type allele or the replacement of a wild type allele with a mutant allele). In some instances, the subject method is for the characterization of a gene function across different cell types. In some instances, 2, 3, 4, 5, 6, 7, 8, 9, 10, 15, 20, 25, 30, 35, 40, 45, 50, 55, 60, 65, 70, 75, 80, 85, 90, 95, or 100 or more different cell types are used with the subject methods. In applications where more than one cell type is used, an individual partition may contain a single cell, a plurality of cells of the same cell type or a plurality of cells of different cell types.

The methods and systems provided herein can be used for altering eukaryotic cells or prokaryotic cells. The eukaryotic cells may be those of or derived from a particular organism, for example, a mammal, including but not limited to human, mouse, rat, rabbit, dog, or non-human primate. Such cells may be isolated as blood or tissue samples from organisms or may be established cell lines. Examples of cell lines that can be used with the subject systems and methods include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr -/-, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

Cells that are have undergone a nucleic acid manipulation event may undergo further processing depending on the application of the method. In screens for a particular phenotype, the cells may be placed under selective conditions for the particular phenotype and the phenotype screened. For example, in screens for growth mutants, cells may be transferred into microwells or suitable partitions that allow for cellular growth and the presence or absence of growth is assayed after a determined period of time. Screening of the phenotype may be performed using any suitable techniques including, for example, fluorescence, luminescence and high-content imaging techniques. See, e.g., Hasson et al., Nature 504: 291-295 (2013); Neumann et al., Nature Methods 3: 385-390 (2006); and Moffat et al., Cell 124: 1283-1298 (2006). In cases where the phenotype is a cell autonomous phenotype, the phenotype may be selectable by cell sorting as fluorescence or cell surface markers. The nucleic acid mutations attributable to the desired phenotype may be identified by any suitable method. In some cases, next-generation sequencing techniques can be used to determine the nucleic acid manipulation. As discussed herein, detectable labels may also be used to track particular nucleic acid mutations. For example, fluorescent or oligonucleotide “barcode” labels may be used to track particular nucleic acid manipulations associated with particular phenotypes of interest.

### III. DEVICES, SYSTEMS AND KITS

Also provided herein are the microfluidic devices used for partitioning target cells with capsules carrying nucleic acid manipulation reagents as described above. Such microfluidic devices can comprise channel networks for carrying out the partitioning process like those set forth in FIG. 1. Examples of particularly useful microfluidic devices are described in U.S. Provisional Patent Application No. 61/977,804, filed Apr. 4, 2014. Briefly, these microfluidic devices can comprise channel networks, such as those described herein, for partitioning cells into separate partitions, and co-partitioning such cells with nucleic acid manipulation reagents, e.g., disposed on beads. These channel networks can be disposed within a solid body, e.g., a glass, semiconductor or polymer body structure in which the channels are defined, where those channels communicate at their termini with reservoirs for receiving the various input fluids, and for the ultimate deposition of the partitioned cells, etc., from the output of the channel networks. By way of example, and with reference to FIG. 1, a reservoir fluidly coupled to channel 102 may be provided with an aqueous suspension of cells 114, while a reservoir coupled to channel 104 may be provided with an aqueous suspension of beads 116 carrying the nucleic acid manipulation reagents. Channel segments 106 and 108 may be provided with a non-aqueous solution, e.g., an oil, into which the aqueous fluids are partitioned as droplets at the channel junction 112. Finally, an outlet reservoir may be fluidly coupled to channel 110 into which the partitioned cells and beads can be delivered and from which they may be harvested. As will be appreciated, while described as reservoirs, it will be appreciated that the channel segments may be coupled to any of a variety of different fluid sources or receiving components, including tubing, manifolds, or fluidic components of other systems.

Also provided are systems that control flow of these fluids through the channel networks e.g., through applied pressure differentials, centrifugal force, electrokinetic pumping, capillary or gravity flow, or the like.

Also provided herein are kits for high throughput alteration of nucleic acids in a plurality of target cells. The kits may include one, two, three, four, five or more, up to all of partitioning fluids, including both aqueous buffers and non-aqueous partitioning fluids or oils, nucleic acid manipulation reagents releasably associated with capsules (e.g., beads), as described herein, microfluidic devices, addition reagents for cellular uptake of the nucleic acid manipulation reagents, as well as instructions for using any of the foregoing in the methods described herein.

### IV. APPLICATIONS

The subject systems and methods provided herein can be used to produce a large population of single cells that each carry the same mutation or different mutations. For example, the system and methods can be used to carry out high throughput genome scale screens. Such screens can be carried out with libraries that are able to perturb a plurality of target nucleic acids in the target cell genome. In some instance where a CRISPR system is used, the nucleic acid manipulation reagents include a guide RNA library spans the target cell genome. In some instances, the nucleic acid manipulation reagents include 10¹, 10², 10³, 10⁴, 10⁵, 10⁶, 10⁷, 10⁸, 10⁹, or 10¹⁰ or more different guide RNAs that target an equal number of different target nucleic acids in the target cell genome. Such methods can be used, for example, in positive screens to identify perturbations that confer resistance to a drug, toxin or pathogen. In such instances, a drug, toxin or pathogen is introduced to each cell containing partition after the cell has undergone a target nucleic acid alteration. Cells that continue to grow in the presence of the drug, toxin, or pathogen are categorized as containing a protective mutation attributable to an alteration of a target nucleic acid. Such target nucleic acid attributable to the phenotype of interest are identified and further characterized, for example, using high throughput sequencing techniques (e.g., next generation sequencing techniques) as discussed above.

In some instances the subject system and methods are used for negative selection under a chosen selective pressure. For instance, in some applications, cells that have undergone nucleic acid editing are selected for a cellular function of interest, for example, extended growth. In such cases, depleted cells that are unable to grow are classified as carrying reagents that target nucleic acids essential for cell proliferation. Such negative screens can identify gene perturbations that selectively target cancer cells having known oncogenic mutations. Genes identified in such a negative screen can serve as possible cancer drug targets.

The subject systems and methods can also be used for the large scale production of cells containing an alteration in a gene of interest. For example, the subject system and methods can be used to efficiently create a large number of cells that contain an alteration in a known oncogene. Such mutant cells can then subsequently be used to screen for other genes that can inhibit growth. The identification of genes that are critical growth are putative drug targets. In some cases, the method is for the production of cells that contain a mutation in a target nucleic acid that affects a biological pathway of interest. Such cells can subsequently be used to identify other genes in the biological pathway. The methods can also be used to repair a mutant gene of interest. For example, the method can be used to replace a mutant allele with a wild type allele in cells isolated from a subject having a disease that is associated with the mutant allele. The repaired cells can then be transplanted back into the subject as part of a treatment for the disease.

The subject systems and methods can also be used for the large scale production of non-human transgenic animals or plants. In some cases, the subject methods can be used to produce transgenic animal that is a mammal, such as a mouse, rat, or rabbit. The subject methods can also be used in the large scale production of crops that contain a nucleic acid mutation of interest, for example, drought resistant crops. See, e.g., Lawlor, 64(1):83-108 (2013), which is herein incorporated by reference in its entirety for all purposes, and particularly for all teachings relating to mutations that confer drought resistance.

The systems and methods provided herein may be used to create a plant, an animal or cell that may be used as a disease model. For instance, the subject methods provided herein may be used to create an animal or cell that may comprise a modification in one or more target nucleic acids associated with a disease, or an animal or cell in which the expression of one or more target nucleic acids associated with a disease are altered. Such target nucleic sequences may encode a disease associated protein sequence or may be a disease associated control sequence. Such disease models can be used to study the development and/or progression of the disease using criteria commonly used for study the disease. Such a disease model is also useful for studying the effect of a pharmaceutically active compound on the disease.

### EXAMPLES

Genome Wide Screen for Tumor-Enhancers and Suppressors

Human KBM7 CML cells are screened for mutations that function in DNA mismatch repair (MMR) using the systems and methods provided herein and CRISPR/Cas9 reagents. In the presence of the nucleotide analog 6-thioguanine (6-TG), MMR-proficient cells are unable to repair 6-TG induced lesions and arrest at the G2-M cell cycle checkpoint, while MMR-defective cells do not recognize the lesions and continue to divide.

CRISPR/Cas9 reagents for creating genome wide mutations are constructed. A guide RNA (gRNA) library containing 50,000 different gRNAs that target over 5,000 different KBM7 genes is constructed. The gRNA library contains 10 gRNAs for each of the 5,000 genes. Each gRNA also includes an oligonucleotide barcode sequence for identification: gRNAs that target the same gene have the same barcode sequence, whereas gRNAs that target different genes have different sequences. gRNAs from the gRNA library are chemically cross-linked to gel beads such that each bead contains gRNAs that target the same gene.

Gel beads carrying the gRNAs and Cas9 nuclease are introduced into droplet partitions containing KBM7 CML cells using a microfluidic device as shown schematically in FIG. 1. First partitions containing a full set of CRISPR/Cas9 reagents are produced, each first partition is a droplet, containing a Cas9 nuclease and a gel bead carrying a guide RNA. Each first partition is then partitioned into a second partition containing transfection reagents. Each second partition is then partitioned into a third partition containing a single cell and a chemical reagent that dissolves the gel beads. In the third partition, the CRISPR/Cas9 reagents are released from the beads and taken up by the KBM7 cell, thereby allowing gene editing in the KBM7 cell.

Cells from each partition are then pooled and grown in the presence of 6-TG. Cells that are capable of multiplying under these conditions presumably contain disruptions in genes that affect MMR. Such cells are isolated and sequenced to identify genes that are involved in MMR. Sequencing is facilitated by the unique barcode identifiers included in each gRNA.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

1. A method of delivering reagents into individual cells, the method comprising:
(a) providing a plurality of capsules, wherein the capsules comprise reagents for altering expression of at least one gene product in a cell;
(b) delivering the capsules into discrete partitions, wherein the discrete partitions further comprise individual cells;
(c) causing the capsules to release their contents into the discrete partitions under conditions enabling uptake of the reagents by the individual cells,
thereby delivering the reagents into the individual cells.

2. The method of claim 1, wherein the reagents for altering expression of at least one gene product encoded by a DNA molecule comprise:
(i) a first regulatory element operably linked to at least one nucleotide sequence encoding a CRISPR system guide RNA that hybridizes with a target sequence in the DNA molecule within the individual cells,
(ii) a second regulatory element operably linked to a nucleotide sequence encoding an RNA-guided nuclease or an RNA-guided nuclease fusion protein.
wherein components (i) and (ii) are located on same or different vectors, and, wherein the RNA-guided nuclease and the guide RNA do not naturally occur together.

3. The method of claim 2, wherein the second regulatory element is operably linked to a nucleotide sequence encoding a RNA-guided nuclease, whereby the guide RNA targets the target sequence and the RNA-guided nuclease cleaves the DNA molecule, whereby expression of the at least one gene product is altered.

4. The method of claim 3, wherein the reagents for altering gene expression further comprise a donor nucleic acid that is inserted into the DNA molecule following cleavage of the DNA molecule by the RNA-guided nuclease.

5. The method of claim 2, wherein the second regulatory element is operably linked to a nucleotide sequence encoding a deactivated RNA-guided nuclease, whereby the guide RNA targets the target sequence and the deactivated RNA-guided nuclease interferes with the transcription of a nucleic acid encoding the at least one gene product, whereby expression of the at least one gene product is altered.

6. The method of claim 2, wherein the second regulatory element is operably linked to a nucleotide sequence encoding a RNA-guided nuclease fusion protein, whereby the guide RNA targets the target sequence and the RNA-guided nuclease fusion protein interferes with the expression of the at least one gene product, whereby expression of the at least one gene product is altered.

7. The method of claim 6, wherein the RNA-guided nuclease fusion protein comprises a deactivated RNA-guided nuclease and a transcription activator or a transcription repressor.

8. The method of claim 6, wherein the nuclease fusion protein comprises a deactivated RNA-guided nuclease and an epigenetic modifier.

9. The method of claim 1, wherein the RNA-guided nuclease is a Cas9 protein or a Cpf1 protein.

10. The method of claim 1, wherein the capsules are configured to release their contents upon the application of a stimulus.

11. The method of claim 10, wherein the stimulus is selected from a chemical stimulus, an electrical stimulus, a thermal stimulus, a magnetic stimulus, a change in pH, a change in ion concentration, reduction of disulfide bonds, a photostimulus, and combinations thereof.

12. The method of claim 11, wherein the stimulus is a thermal stimulus.

13. The method of claim 1, wherein the plurality of capsules comprises about 100-100,000 different reagents for altering expression of at least one gene product, such that different individual cells receive different reagents.

14. The method of claim 1, wherein the capsules further comprise one or more additives for compatibility of the capsules or their contents with the individual cells.

15. The method of claim 14, wherein the one or more additives comprises a transfection agent.

16. The method of claim 1, wherein the reagents for altering expression of at least one gene product further comprise oligonucleotides that comprise a nucleic acid barcode sequence, and wherein different individual cells receive different nucleic acid barcode sequences.

17. The method of claim 2, wherein the reagents for altering expression of at least one gene product further comprise a pair of Cas9 nickases or Cas9 fusion proteins that improve specificity of the CRISPR system as compared to when RNA-guided nucleases are used.

18. The method of claim 2, wherein the target sequence has few or no close relatives within the cellular genome.

19. The method of claim 2, wherein the reagents for altering expression of at least one gene product further comprise agents that increase frequency of homologous recombination in the cell by repressing genes involved in non-homologous end-joining (NHEJ) pathway.

20. The method of claim 19, wherein the agents comprise a Cas9 nuclease or a nuclease-null Cas9 protein encoded with the at least one nucleotide sequence encoding a CRISPR system guide RNA.

21. The method of claim 2, wherein the guide RNA further comprises a spacer that is identical to a targeted protospacer sequence within the cell's genome.

22. The method of claim 2, wherein the second regulatory element is an inducible promoter.

23. The method of claim 22, wherein the inducible promoter is selected from the group consisting of a light-inducible, a heat-inducible and a chemical inducible promoter.

24. A method for delivering a reagent to a cell, the method comprising:
(a) providing the reagent releasably coupled to a microcapsule;
(b) separating the microcapsule into a discrete partition, wherein the discrete partition further comprises an individual cell; and
(c) releasing the reagent under conditions that enable uptake of the reagent into the cell.

25. The method of claim 24, wherein the reagent comprises a vector encoding at least one of a RNA-guided nuclease, Clustered Regularly Interspaced Short Palindromic Repeats (CRISPRs), or CRISPR guide RNA capable of hybridizing with one or more target sequences in a DNA molecule of the cell, and one or more condition-inducible promoters.

26-44. (canceled)

45. A method for altering gene expression in a plurality of cells, the method comprising:
(a) providing a plurality of capsules, wherein capsules comprise reagents for altering expression of at least one gene product, the reagents comprising an engineered, non-naturally occurring Clustered Regularly Interspaced Short Palindromic Repeats (CRISPR) system comprising one or more vectors comprising:
(i) a first regulatory element operably linked to at least one nucleotide sequence encoding a CRISPR-Cas system guide RNA capable of hybridizing with a target sequence in the DNA molecule of the cell, and
(ii) a second regulatory element operably linked to a nucleotide sequence encoding a RNA-guided nuclease protein, wherein components (i) and (ii) are located on same or different vectors of the system,
(b) delivering the capsules into discrete partitions containing individual cells;
(c) providing a stimulus to cause the capsules to release their contents under conditions such that reagents are delivered into the individual cells,
wherein subsequent to application of the stimulus, the guide RNA hybridizes to the target sequence and the RNA-guided nuclease protein cleaves the DNA molecule containing the target sequence, whereby expression of the at least one gene product is altered.

46. (canceled)

47. A method for altering gene expression in a plurality of cells, the method comprising:
(a) providing a plurality of capsules, wherein capsules comprise reagents for altering expression of at least one gene product, the reagents comprising an engineered, non-naturally occurring Clustered Regularly Interspaced Short Palindromic Repeats (CRISPR) system comprising one or more vectors comprising:
(i) a first regulatory element operably linked to at least one nucleotide sequence encoding a CRISPR-Cas system guide RNA capable of hybridizing with a target sequence in the DNA molecule of the cell, and
(ii) a second regulatory element operably linked to a nucleotide sequence encoding a deactivated RNA-guided nuclease, wherein components (i) and (ii) are located on same or different vectors of the system,
(b) delivering the capsules into discrete partitions containing individual cells;
(c) providing a stimulus to cause the capsules to release their contents under conditions such that reagents are delivered into the individual cells,
wherein subsequent to application of the stimulus, the guide RNA hybridizes to the target sequence and the deactivated RNA-guided nuclease interferes with the transcription of a nucleic acid encoding the at least one gene product, whereby expression of the at least one gene product is altered.

48. A method for altering gene expression in a plurality of cells, the method comprising:
(a) providing a plurality of capsules, wherein capsules comprise reagents for altering expression of at least one gene product, the reagents comprising an engineered, non-naturally occurring Clustered Regularly Interspaced Short Palindromic Repeats (CRISPR) system comprising one or more vectors comprising:
(i) a first regulatory element operably linked to at least one nucleotide sequence encoding a CRISPR-Cas system guide RNA capable of hybridizing with a target sequence in the DNA molecule of the cell, and
(ii) a second regulatory element operably linked to a nucleotide sequence encoding a RNA-guided nuclease fusion protein, wherein components (i) and (ii) are located on same or different vectors of the system,
(b) delivering the capsules into discrete partitions containing individual cells;
(c) providing a stimulus to cause the capsules to release their contents under conditions such that reagents are delivered into the individual cells,
wherein subsequent to application of the stimulus, the guide RNA hybridizes to the target sequence and the RNA-guided nuclease fusion protein interferes with the expression of the at least one gene product, whereby expression of the at least one gene product is altered.

49-60. (canceled)
